(19) United States
(12) Patent Application Publication
(10) Pub. No.: US 2011/0030486 A1
(43) Pub. Date: Feb. 10, 2011

(54) DEVICE FOR GAUGING THE STATUS OF A MATERIAL ESPECIALLY OF OILS OR FATS

(76) Inventors: Jurgen Hall, Rotenbach (DE); Mike Muhl, Freiburg (DE)

Correspondence Address: LACKENBACH SIEGEL, LLP, LACKENBACH SIEGEL BUILDING, 1 CHASE ROAD, SCARSDALE, NY, 10583, US

(21) Appl. No.: 12/669,726

(22) PCT Filed: Jul. 29, 2008
(86) PCT No.: PCT/EP2008/006238

§ 371 Date: Jun. 2, 2010

(30) Foreign Application Priority Data
Aug. 1, 2007 (DE) 10 2007 036 473.5

(51) Int. Cl. G01N 33/03

(52) U.S. Cl. 73/866

(57) ABSTRACT

The invention is a device for gauging the status of a material such as a fat or an oil. The device comprises: a housing; a hollow connecting element; a substrate, attached at the opposite end of the hollow connecting element; a sensor, proximate the substrate, for measuring an electrical property of the material being tested; a protective coating, wherein the protective coating covers the sensor and can be applied using either a thin-film or a thick-film technology; and, an electronic evaluation unit connected via at least one electric line to the sensor; and, is arranged proximate the housing and/or proximate the end of the connecting element that faces the housing.

### CROSS REFERENCE TO RELATED APPLICATIONS

This application claims priority from PCT Patent Application Ser. No. PCT/EP 2008/006238, filed Jul. 29, 2008, the entire contents of which is herein incorporated fully by reference, which in turn claims priority form German Patent Application Serial No. 10 2007 036 473.5, filed Aug. 1, 2007.

### BACKGROUND OF THE INVENTION

1. Field of the Invention

The present invention relates to a device for gauging the status of a material, especially of oils or fats. More particularly, the present invention is a device for gauging the status of oils or fats, and which has increased mechanical stability and is easier to clean. In particular, the present invention ensures the prevention of inaccurate measured values, especially those caused by material residues.

2. Description of the Related Art

The related art involves oils or fats which are frequently used in preparing foods. In this context, a fat is understood specifically as the solid form of an oil. Fats or oils of this type are frequently used not just a single time, but over an extended period of time, for example in deep fat fryers, to fry successive batches of various foods. The temperature of the oil or fat in the deep fat fryer is approximately 130° C. to 180° C. At these temperatures, the fat or oil is subject to aging processes, such as oxidation caused by atmospheric oxygen; this results in numerous chemical decomposition products, such as aldehydes, ketones and polymers, which can have an adverse effect on flavor and may even be hazardous to the health.

It is therefore important to replace these oils or fats, especially oils or fats used in deep frying, regularly and on schedule, while ensuring that the oils or fats are not replaced too early, i.e., when the oils or fats are still usable, or too late, i.e., when the oils or fats have already become largely decomposed. For this purpose, devices for gauging the status of oils or fats are used, for example, which measure the electrical properties of the oils or fats. Electrical properties, especially the dielectric constant of the oils or fats, are a reliable measurement of the degree of decomposition of the fat or the oil.

DE 10 2004 016 955 A1, DE 10 2004 016 957 A1 and DE 10 2004 016 958 A1 each disclose a device for gauging the status of oils or fats, comprising a housing, a hollow connecting element connected to this, and a substrate attached to the opposite end of the connecting element and configured to hold a sensor for measuring an electrical property of the material being tested, wherein the sensor is connected via at least one electric line to an electronic evaluation unit, which is positioned in the vicinity of the housing and/or the end of the connecting element that faces the housing. The substrate can be submerged with the sensor in the oil or fat and is capable of determining its dielectric constant. In this process, the sensor comes into direct contact with the hot oil or fat and is therefore exposed to extreme stress. To protect the sensor from contact with the base or the walls of the measuring vessel containing the oil or fat, the aforementioned publications disclose a U-shaped protective element, which is configured to extend around the rim of the flat substrate, thereby protecting the sensor from impacts.

DE 200 23 601 U1, DE 101 63 760 A1 and DE 20 2005 007 144 U1 also each disclose a device for gauging the status of a material comprised of oils or fats, in which a sensor is positioned at an unattached end of a connecting element on a substrate, which can be submerged in the material being tested. In this case the sensor can be encased in a shielding or covering, which is intended to protect the sensor from damage caused by impacts against the interior walls of the measuring vessel, but can also provide at least partial shielding against parasitic capacitances, as is described in DE 101 63 760 A1, for example, so that the functioning of the sensor is not impaired. The shields disclosed in these publications also protect the sensor only against the stress of impacts, with the shields being configured either like a stirrup or, as described in DE 101 63 760 A1, as spatially encompassing the sensor, however the shielding is configured such that the hot oil or fat passes through the shielding and washes directly around the sensor.

What is not appreciated by the prior art is The sensors that are used are generally embodied as capacitors, especially as interdigital capacitors, comprised of intermeshed fine gold wires or gold tracks, which can especially be applied via printing or vapor deposition. These generally have relatively low mechanical stability. Mechanical stress, such as occurs, for example, when cleaning the sensor by rubbing it with a paper towel, can alter the thickness of the gold tracks. Added to this is the problem that oil or fat residues can settle between the structures, resulting in an increase in the basic capacitance of the sensor and thus inaccurate test results. If the oil or fat residues are not removed, these will continue to age, causing the results obtained by the sensor to continuously change.

Accordingly, there is a need for an improved device for gauging the status of a material, especially of oils or fats, which has increased mechanical stability and which is easier to clean.

### ASPECTS AND SUMMARY OF THE INVENTION

An aspect of the present invention is to provide a device for gauging the status of a material, especially of oils or fats, which has increased mechanical stability and which is easier to clean. In particular, it is advantageous to ensure the prevention of inaccurate measured values, especially caused by material residues.

The present invention relates to a device for gauging the status of a material such as a fat or an oil. The device comprises: a housing; a hollow connecting element; a substrate, attached at the opposite end of the hollow connecting element; a sensor, proximate the substrate, for measuring an electrical property of the material being tested; a protective coating, wherein the protective coating covers the sensor and can be applied using either a thin-film or a thick-film technology; and, an electronic evaluation unit connected via at least one electric line to the sensor; and, is arranged proximate the housing and/or proximate the end of the connecting element that faces the housing.

According to the invention, the sensor is covered by a protective coating, which fully protects the sensor from any direct contact with the material being tested. The sensor no longer comes into direct contact with the hot material being tested and is therefore subject to lower stress. This extends the lifespan of the sensor. The protective coating also prevents residues of the material being tested from settling into cavities of the sensor, as these cavities are covered by the protective coating, so that the sensor does not become as soiled, and inaccurate test results caused by residue of tested material are prevented. This also makes the device easier to clean. Finally, the stability of the device is increased, because the sensor, especially the gold wires or the gold tracks of the sensor, if present, are protected against mechanical stresses.

The protective coating preferably covers the substrate, including the sensor, because a protective coating of this type can be more favorably produced. In particular, this configuration has no edges between the protective coating and the substrate to which tested material could adhere, and the electric lines between the sensor and the electronic evaluation unit are protected.

The sensor preferably detects the dielectric constant of the material being tested, as this correlates to the aging status of the material being tested, especially the oil or fat.

In a particularly preferred embodiment of the invention, the sensor is embodied as a capacitor, preferably as an interdigital capacitor, because when a capacitor is used, the dielectric constant can be particularly easily measured. An interdigital capacitor enables an especially reliable measurement of the dielectric constant and is also less sensitive to interference factors.

To avoid excessively decreasing the sensitivity of the sensor, the protective coating is preferably less than 10 μm thick, preferably less than 1 μm thick.

To increase the mechanical stability of the gauge, in a preferred embodiment of the invention, the hardness of the protective coating is greater than the hardness of gold, since the structures of the sensor are ordinarily made of gold conductor tracks, and the protective coating may not be softer than the sensor itself, so as not to diminish its mechanical stability.

The protective coating is preferably furnished so as not to affect the capacitive measurement of the sensor. For this reason, the surface resistivity of the protective coating is preferably greater than 1 MΩ, especially greater than 10 MΩ, in order to ensure a low conductivity of the protective coating. The protective coating further preferably has a permittivity of less than 10, with the permittivity of the protective coating especially preferably being lower than the permittivity of the material being tested. In a further preferred embodiment, the temperature dependence of the permittivity of the protective coating is lower than the temperature dependence of the permittivity of the material being tested, in order to avoid impacting the functioning of the sensor.

In an advantageous embodiment of the invention, the protective coating has the same or a lower roughness than the substrate, so as to avoid a deterioration of the roughness of the substrate. A lower roughness and thus a smooth surface prevent abrasive wear and the accumulation of test material residues in small cavities. The substrate is preferably made of an insulator, especially a ceramic, in order to insulate the sensor tracks in relation to one another. A ceramic has a roughness of approximately 0.2 μm to 0.8 μm, so that especially preferably, the protective coating has a roughness lower than 0.8 μm, especially lower than 0.2 μm.

The protective coating is preferably resistant to oxidation, at least resistant to oxidation at the usual temperatures at which it is used, in order to ensure that the protective coating will undergo no chemical changes over its period of use which might impair the functioning of the sensor.

Preferably the protective coating, at least at the usual temperatures at which it is used, also will not react in any way with the tested material itself, with decomposition products of the tested material, or with cleaning agents, especially with alkaline cleaning agents, in order to ensure that during its period of use and at the usual temperatures at which it is used no chemical changes to the protective coating will occur which could impair the functioning of the sensor, and to ensure that the gauge can be cleaned with suitable cleaning agents after use.

In one particularly preferred embodiment of the invention the protective coating has an oleophobic surface, which is achieved, for example, by using a corresponding material for the protective coating or by using surface structures on the protective coating. This reduces the adhesion of the tested material to the surface, making the gauge much easier to clean.

Preferably a metal oxide, a metal nitride, a metal carbide, an amorphous carbon layer or a mixture of at least two of these compounds is used for the protective coating. The protective coating is preferably made of oxides of silicon, aluminum, titanium or zirconium, of nitrides of titanium or silicon, or of carbides of titanium or silicon, or of mixtures of these compounds. Materials of this type do not affect the functioning of the sensor, do not undergo chemical change over the period of use and at the usual temperatures of use, and possess sufficient hardness and a sufficiently smooth surface to ensure the mechanical stability and ease of cleaning of the sensor.

The protective coating is preferably applied using thin- or thick-film technology, for example via physical vapor deposition (PVD), chemical vapor deposition (CVD), screen printing, spin coating, dip coating, spraying or other processes.

The above, and other aspects, features and advantages of the present invention will become apparent from the following description read in conduction with the accompanying drawings, in which like reference numerals designate the same elements.

### BRIEF DESCRIPTION OF THE DRAWINGS

FIG. 1 is a drawing of the front view of a first embodiment of the device of the invention.

FIG. 2 is an enlarged view of the lower area of the device of FIG. 1, which will be submerged in the material to be tested.

FIG. 3 is a longitudinal section of the lower area of the device of FIG. 2.

### DETAILED DESCRIPTION OF THE PREFERRED EMBODIMENTS

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, and below may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices.

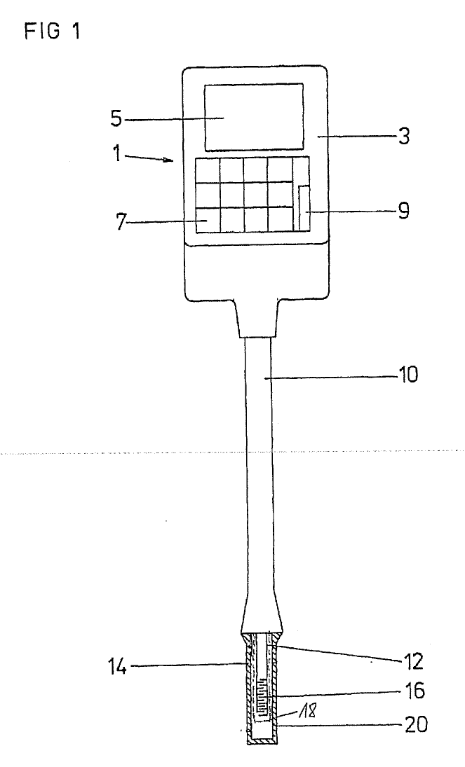

FIG. 1 shows a gauge 1 of the invention for gauging the status of a material, especially of oils or fats, which has a housing 3 at its upper end. The housing has a display 5 for displaying measured values. The display 5 is preferably embodied as an LCD display, and can be switched between a graphic display, which indicates the measured values by color gradations, and a numerical display. For inputting control commands, a keyboard 7 is provided, via which commands can be sent to the central control unit (not shown). The keyboard 7 is preferably embodied as a touch screen. The housing 3 can also preferably have an interface 9, which can be used to communicate with external computers. The gauge 1 is preferably adapted to perform a self-calibration. When the gauge 1 is in use, the housing 3 also serves as a handle for the operator.

A hollow connecting element 10 extends downward out of the housing 3, and is made of sufficient length out of a material with poor thermal conductivity, so that the sensitive electronic evaluation unit (not shown) of the gauge 1, which is located in the vicinity of the housing 3 and/or in the area of the connecting element 10 close to the housing 3, is adequately protected from the heat of the oil or fat being tested. This measure also ensures that the operator can perform the measurements safely. The connecting element 10 is preferably made of stainless steel, which, in addition to its low thermal conductivity, is also suitable due to its unrestricted use in the food services industry. The connecting element 10 is configured as a tubular component, for example, and is able to accommodate electric lines 12, which run in the interior of the connecting element 10. The electric lines 12 are arranged on at least one flat substrate 14, which is characterized by electrical insulation properties, for example a substrate 14 made of ceramic material.

In the lower area of the substrate 14, a sensor 16 for measuring the electrical properties of the oil or fat is arranged, the measured values of which are fed via the electric lines 12 on the substrate 14 to the electronic evaluation unit. A protective means 20 can be attached around the lower area of the substrate 14 to protect the sensor 16 from contact with the base or the walls of the measuring vessel. In the present case, the protective means 20 is configured as a rim that surrounds the flat substrate 14 and is connected to the connecting element 10, thus it is embodied substantially as a U-shaped bracket.

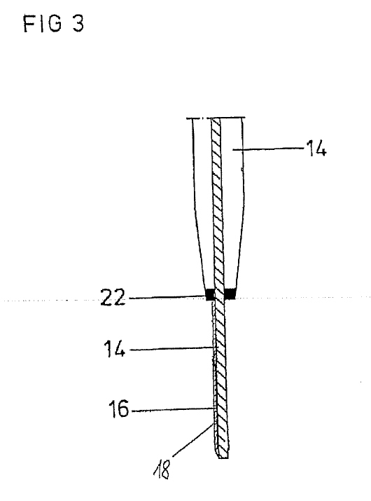

The intermediate space between substrate 14 and connecting element 10 is insulated and sealed at one point via suitable sealing means 22, as is apparent in FIG. 3. There, a suitable adhesive 22, such as a silicone adhesive, is injected into the intermediate space between substrate 14 and connecting element 10 in the lower end area of the connecting element 10, thereby preventing them from coming into direct contact with one another and thus insulating them from one another. At the same time, the adhesive 22 serves to seal off the connecting element 10, so that no oil or fat can penetrate into the interior of the connecting element 10. The configuration of the adhesive surface must be safe from water entrapment, as otherwise the risk of explosion exists and a potential contamination of the material being tested by cleaning agent cannot be ruled out. The substrate 14, as a single-piece element, can extend up to the electronic evaluation unit, however it may also be uncoupleable, with a series connection of multiple connectors. This configuration offers advantages especially in terms of the thermal resistance of the electronic evaluation unit.

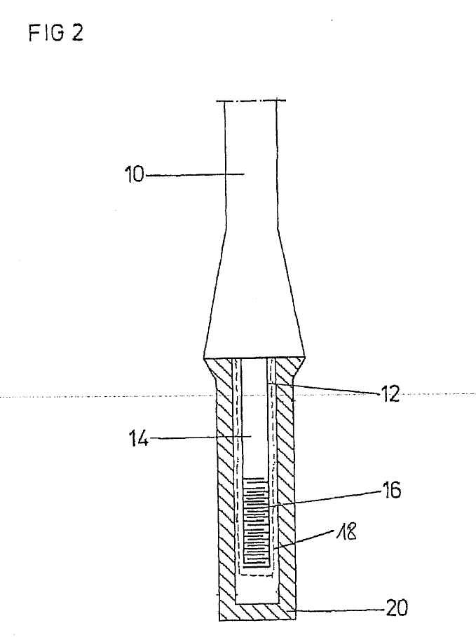

In FIG. 2, the lower area of the connecting element 10 and the substrate 14, which is to be submerged in the material being tested, are shown in an enlarged view. The sensor 16 for measuring the dielectric constant comprises a capacitor, which measures the dielectric constant of the oil or fat. It is preferably embodied as an interdigital capacitor, which is comprised of fine, enmeshed gold wires or gold tracks, which are especially applied via printing or vapor deposition, and each of which transitions into one of the electric lines 12 that lead to the electronic evaluation unit. The lines 12 are comprised of a fine layer of gold or copper extending from the substrate 14, wherein the layer is printed directly onto the ceramic component.

The sensor 16 is covered by a protective coating 18. The protective coating 18 is embodied such that it covers at least the sensor 16 in such a way that the sensor 16 has no direct contact with the material being tested, into which the device 1 will be submerged. As the figures illustrate, the protective coating 18 also preferably covers the electric lines 12 completely, so that these also do not come into direct contact with the material being tested. It is also possible for the protective coating 18 to be arranged not only on the front side of the substrate 14 where the sensor 16 is arranged, but for the entire lower area of the substrate 14, on the front and rear sides, to be covered with the protective coating 18.

The thickness of the protective coating 18 is less than 1 μm. Further, the hardness of the protective coating is greater than that of the material of the electric lines 12, in other words in this case greater than the hardness of gold. In this manner, the mechanical stability of the device 1, especially the stability of the gold wires or the gold tracks, is increased.

To ensure that the protective coating 18 will not affect the capacitive measurement by the sensor 16, the protective coating 18 must have stable electrical properties. For this reason, the surface resistivity of the protective coating 18 is greater than 10 MΩ. Additionally, the protective coating 18 has a permittivity of less than 10. Because oils and fats have a permittivity of approximately 3, the protective coating 18 preferably has a permittivity that is lower than the permittivity of the material being tested, therefore lower than 3. Additionally, the temperature dependence of the permittivity of the protective coating 18 is lower than the temperature dependence of the permittivity of the material being tested, in order to ensure that the capacitive measurement of the sensor 16 is not affected.

To prevent any residue of test material from settling in structures in the protective coating 18, the protective coating has a roughness of less than 0.2 μm. The protective coating is made of oxides of silicon, aluminum, titanium or zirconium, or of nitrides of titanium or silicon, or of carbides of titanium or silicon, or of mixtures of these compounds. A protective coating 18 made of these materials is correspondingly resistant to oxidation, to reaction with the test material or with decomposition products of the test material, and to cleaning agents, especially to alkaline, especially highly alkaline cleaning agents. Additionally, a protective coating 18 made of this material will adhere well to the surface of the substrate 14 and the surface of the sensor 16. Moreover, the protective coating 18 can be applied to the substrate 14 and the sensor using conventional processes such as PVD, CVD, screen printing, spin coating, dip coating, spraying, etc. Additionally, the protective coating can be applied using thin- or thick-film technologies.

In the claims, means or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

1-16. (canceled)

17. A device for gauging the status of a material to be tested, especially an oil or a fat material, said device comprising:
(a) a housing;
(b) a hollow connecting element connected to said housing;
(c) a substrate, attached at an opposite end of said hollow connecting element;
(d) a sensor, proximate said substrate, said sensor for measuring an electrical property of said material being tested;
(e) a protective coating, wherein said protective coating is configured to cover said sensor; and
(f) an electronic evaluation unit, said electronic evaluation unit connected via at least one electric line to said sensor; and said electronic evaluation unit arranged proximate the end of said connecting element facing said housing.

18. The device of claim 17, wherein:
said protective coating covers said substrate, including said sensor.

19. The device of claim 17, wherein:
said sensor is capable of measuring a dielectric constant of said material to be tested.

20. The device of claim 17, wherein:
said sensor is embodied as one of a capacitor and an interdigital capacitor.

21. The device of claim 17, wherein:
said protective coating has a thickness of less than 10 μm.

22. The device of claim 21, wherein:
said protective coating has a thickness of less than 1 μm.

23. The device of claim 17, wherein:
a hardness of said protective coating is greater than a hardness of gold.

24. The device of claim 17, wherein:
a surface resistivity of said protective coating is greater than 1 MΩ.

25. The device of claim 24 wherein:
said surface resistivity of said protective coating is greater than 10 MΩ

26. The device of claim 17, wherein:
a permittivity of said protective coating is less than 10; and
said permittivity of said protective coating is lower than the permittivity of said material being tested.

27. The device of claim 26, wherein:
a temperature dependence of said permittivity of said protective coating is lower than a temperature dependence of said permittivity of said material to be tested.

28. The device of claim 17, wherein:
said protective coating has the same or a lower roughness than a roughness of said substrate, wherein said roughness of said protective coating is preferably less than 0.8 μm.

29. The device of claim 28, wherein:
said the roughness of said protective coating is preferably less than 0.2 μm.

30. The device of claim 17, wherein:
said protective coating is resistant to oxidation; and
said protective coating is substantively non-reactive with any one of the group comprising:
i. said material being tested;
ii. decomposition products of said material being tested; and
iii. cleaning agents.

31. The device of claim 17, wherein:
said protective coating comprises an oleophobic surface.

32. The device of claim 17, wherein:
said protective coating comprising:
a compound selected from the group comprising:
iv. a metal oxide;
v. a metal nitride;
vi. a metal carbide;
vii. an amorphous carbon layer; and
viii. a mixture of at least two of said compounds listed in (a) through (d).

33. The device of claim 17, wherein:
said protective coating is comprised of at least one of an oxide, a nitride, and a carbide, wherein said at least one is selected from:
oxides selected from the group comprising: silicon; aluminum; titanium; and zirconium; nitrides selected from the group comprising: nitrides of titanium and nitrides of silicon; and carbides selected from the group comprising: carbides of titanium and carbides of silicon.

34. (news) The device of claim 17, wherein:
said protective coating is comprised of carbides selected from the group comprising: titanium and silicon.

35. The device of claim 17, wherein:
said protective coating is comprised of compounds selected from the group comprising mixtures of at least two compounds, said compounds selected from the group comprising: silicon oxide, aluminum oxide, titanium oxide, titanium nitride, titanium carbide, zirconium oxide, silicon nitride; and silicon carbide.

36. A device for gauging the status of a material, particularly an oil or a fat material, said device comprising:
(a) a housing;
(b) a hollow connecting element connected to said housing; and
(c) a substrate, attached at the opposite end of said hollow connecting element and configured to hold a sensor;
(d) said sensor being proximate said substrate, said sensor being operative for measuring an electrical property of said material being tested, and wherein said sensor is covered by a protective coating;
(e) said sensor is capable of measuring a dielectric constant of said material to be tested and being embodied as one of a capacitor and an interdigital capacitor;
(f) said coating for said sensor having a thickness of less than 10 μm; a hardness that is greater than a hardness of gold, a surface resistivity of greater than 1 MΩ; and a permittivity of said protective coating is less than 10; and
(g) an electronic evaluation unit, said electronic evaluation unit connected via at least one electric line to said sensor; and is arranged proximate said housing.

37. A device for gauging the status of a material, said device comprising:
(a) a housing;
(b) a hollow connecting element connected to said housing; and
(c) a substrate, attached at the opposite end of said hollow connecting element;
(d) a sensor, proximate said substrate, said sensor for measuring an electrical property of said material being tested;
(e) a protective coating, wherein said protective coating:
(i) covers said sensor;
(ii) is resistant to oxidation; and
(iii) has an oleophilic surface; and
(f) an electronic evaluation unit, said electronic evaluation unit connected via at least one electric line to said sensor; and is arranged proximate the end of said connecting element that faces said housing.
